(19) United States
(12) Patent Application Publication
(10) Pub. No.: US 2017/0241934 A1
(43) Pub. Date: Aug. 24, 2017

(54) Matric Potential Sensor

(71) Applicant: Delta-T Devices Limited, Cambridge (GB)

(72) Inventors: Martin Goodchild, Cambridge (GB); Malcolm Jenkins, Cambridge (GB); Chris Saywell, Cambridge (GB)

(21) Appl. No.: 15/503,575

(22) PCT Filed: Aug. 14, 2015
(86) PCT No.: PCT/GB2015/052345

§ 371 Date: Feb. 13, 2017

(30) Foreign Application Priority Data
Aug. 15, 2014 (GB) 1414492.7

(51) Int. Cl. G01N 27/22; A01G 25/16; G01N 33/24

(52) U.S. Cl. CPC G01N 27/225; G01N 33/246; A01G 25/167

(57) ABSTRACT

This invention relates to a matric potential sensor (100). Specifically, but not exclusively, the invention may relate to a sensor containing micro-lattice matrix portions (104) arranged to take up water. The matric potential sensor (100) comprises sensing electronics (120) arranged to measure a change in a signal injected into a matrix via one or more sensing elements(102), wherein the matrix comprises at least one portion of a substantially open-cell hydrophilic micro-lattice matrix (104) arranged to absorb moisture from a medium into which, in use, it is inserted.

This invention relates to a matric potential sensor. Specifically, but not exclusively, the invention may relate to a sensor containing micro-lattice matrix portions arranged to take up water.

Whilst the field of soil-moisture content measurement has been developed for several decades, the field of matric potential measurement is more recent and tries to measure the ability of a medium to take up and retain water. The matric potential of a medium gives a measurement of the medium's attraction for water which may be thought of as the ability of that medium to retain water. As such, knowing the matric potential of a medium gives a useful measurement in determining the availability of water from that medium.

It is convenient to describe the background of this idea in relation to the measurement of soil water content but the invention has wider applicability, and may be of particular benefit when used with soil-free substrates. For example the sensor may be used in any of the non-exhaustive list of substrates and/or media: coir, cotton wool, mineral wool, sand, Rockwool, volcanic material, plant growing media, peat, compost, concrete, building material, pharmaceutical materials and the like. Further, the sensor may also find application for measuring in the, non-exhaustive, list of applications: environmental monitoring, irrigation monitoring, irrigation control, crop yield optimisation, flood control, damp measurement, building subsidence, refuse compost monitoring and drug manufacturing in the pharmaceutical industry.

A medium such as clay which has relatively small pores has an ability to retain water which is higher than that of a coarse medium, such as sandy soils, with relatively large pores. Knowing the matric potential of a medium at a particular water content can be used to determine, for example when to irrigate, etc. and help to avoid over irrigation thereby conserving water. Furthermore, measuring matric potential can provide a soil-type independent assessment of the soil moisture that can be reliably used to avoid water stress conditions that could be detrimental to plant growth and result in reduced crop yields.

Such matric potential measurement systems originally used water-filled tensiometers which suffered from a number of problems, including: being complex to operate; having a limited range of measurement; attraction of the roots of nearby plants due to constant water source; cannot operate in below freezing conditions; need for degassed water.

A medium such as coir has different properties to those of clay. Coir consists of natural fibres, which are hollow. In addition, the mix of fine and coarse fibres provides pore spaces between the fibres.

Water flow through coir is therefore often more rapid than through many other growing media. This can lead to air bubbles becoming trapped around the sensing elements of the sensor if they cannot easily escape from that region, which may lead to inaccurate readings.

As described in L. D. Rivera, L. Crawford, M. van Iersel, and S. K. Dove, “Comparing Hydraulic Properties of Soil-less Substrates with Natural Soils: A More Detailed Look at Hydraulic Properties and Their Impact on Plant Water Availability”, Poster 262, Annual Conference of the American Society for Horticultural Science, Jul. 22-25, 2013, soils and other media have pore size distributions that can be split into three categories:
(i) well graded (even distribution of sizes across a range);
(ii) uniformly-graded (all of around the same size); and
(iii) gap graded (a bimodal pore-size distribution of some small and some large, with a “gap” in the middle).

Gradation affects drainage and hydraulic conductivity. Traditionally, soils are graded using sieves. Media which have smaller pores that are part of larger particles (e.g. coir) cannot be graded in this way, however.

Gap-graded media have hydraulic conductivities that drop very low (500 times lower than agricultural soils—L. Rivera et al. (2013)) at low water potentials (i.e. potentials around −10 kPa). This dramatically lower hydraulic conductivity may lead to zones of depletion around the roots, hindering plant water uptake even when a significant amount of water remains in the medium. This occurs because once the larger pores have been drained, it becomes difficult to move water out of the smaller pores.

The low hydraulic conductivity limits the movement of the remaining water in the substrate even though there is still “available” water. This sharp change in hydraulic conductivity is missed using traditional methods for generating soil moisture release curves, such as pressure plates, hanging water columns, and Tempe pressure cells.

Hydraulic conductivity is also illustrated by: M. Raviv, J. H. Lieth, D. W. Burger and R. Wallach, “Optimization of Transpiration and Potential Growth Rates of ‘Kardinal’ Rose with Respect to Root-zone Physical Properties”, J. Amer. Soc. Hort. Sci. 126(5):638-643, 2001, wherein it is stated that “ . . . in many cases, water flux across the medium/root interface cannot match atmospheric demand for water, even under near-ideal aerial conditions. This is due to a sharp decrease in K (hydraulic conductivity) with decreasing φ (water content)”. This sharp decrease is shown to occur over a range of water potentials of 0 to −10 kPa for the conditions and media (coir and a defined composted fir bark-peat-sand mixture) tested in this paper.

In addition, water potentials in coir growing media are often close to 0 kPa, i.e. the conditions are wet. This can lead to saturation of sensors, preventing accurate readings in wet conditions.

The water retention characteristics of growing media which influence the availability of air and water to plant roots is also illustrated by: J-C Michel, “The physical properties of peat: a key factor for modern growing media”, Mires and Peat, Volume 6, Article 2, 2010, wherein the water retention curves are characterised over the water potential range of 0 to −10 kPa for a selection of peats and peat-free alternative grower substrates, including: Rockwool, Perlite and course sand, clearly identifying regions in soil moisture and tension of high water availability.

Water column potentiometers, such as that described in Canadian patent application number 2,611,196 (Plantcare AG, 2006), can be used to measure soil moisture content.

Water column potentiometers rely on the height of water in the column changing due to changes in water pressure in the soil. Such arrangements are hard to use, particularly if gas enters the system, and are perhaps better suited to a laboratory environment.

According to a first aspect of the invention there is provided a matric potential sensor arranged to measure the matric potential of a medium. The matric potential sensor is suitable for measuring the matric potential of soil-free media. Typically the sensor comprises at least one electrode. The sensor may also comprise a porous material portion, arranged to absorb moisture from the medium into which, in use, it is inserted. Typically the porous material portion is hydrophilic, meaning that it has an affinity for water and is easily wetted. In addition, the porous material portion typically has a substantially open cell micro-lattice structure. Embodiments having these features have an increased hydraulic conductivity of the material.

In some embodiments, the porous material is a plastics material; i.e. a polymeric material. Polyurethane is an example of a suitable polymer for the matrix. Plastics materials offer the advantages of relatively inexpensive material and simple manufacture, although ceramics and composite materials could be used, amongst other materials.

Advantageously, polyurethane foams offer improved water-handling capabilities when used as soil-free growing media. Such materials therefore have favourable properties for use as water potential sensor elements.

Conveniently, the porous material is compliant. Such embodiments are advantageous in that they allow the porous material to conform to the or each electrode, so reducing or removing any gaps between the electrodes and the porous material which could introduce errors in measurements. Such gaps may cause water to be trapped therein which can affect the readings taken by the sensor. Advantageously, this improves contact between the porous material and the electrodes.

In various embodiments, the porous material may be a foam. Advantageously, the porosity and open-cell nature of an initially-formed foam may be increased by reticulation and/or compression prior to incorporation in the sensor. Reticulation and/or compression increases the porosity and makes the structure more open, and controls pore size, which increase hydraulic conductivity. The improved rate of water uptake that the reticulated and/or compressed foam offers reduces the required time for readings to be taken, as the equilibration time of the sensor decreases with increasing hydraulic conductivity. Thus, the porous material may comprise a reticulated and/or compressed foam.

In some embodiments, the porous material may be a foam that may have been generated by a polymer foam replication technique wherein a polymeric foam is used as template and a slurry of the desired material is added, followed by drying and sintering to create a replica of the original polymeric foam. The polymer foam replication technique is particularly advantageous when the desired material for the foam is ceramic, or another material with a substantially higher burn-off temperature than the polymeric foam used as the starting point. Alternatively, the commercial polymeric foam can be dissolved using a suitable solvent known in the art to leave the replicated foam. Thus, the foam may be a ceramic or other non-plastics material.

In other embodiments, the porous material may have been fabricated by 3D printing or other solid freeform fabrication and rapid prototyping technique. Such techniques include techniques such as selective laser sintering or shape deposition modelling each of which may be used to generate a structure similar to that of a reticulated and compressed foam. This structure may be thought of as being a web of interconnected struts.

Such solid freeform fabrication techniques allow control of the porosity, pore size and pore size distribution of the porous material. This is advantageous as it allows the porous material to be tailored to different applications, test media and/or wetness levels.

In addition, such solid freeform fabrication techniques may help to ensure that blocks of the porous material produced to the same template or design are highly similar or identical. This offers the benefit of uniformity in product characteristics and performance. Advantageously, moisture characteristic curves will be identical, within tolerances, for these blocks so reducing the amount of characterisation needed before sale or use. A single moisture characteristic curve may be provided for all blocks generated from the same template or design and provide a high degree of accuracy.

The micro-lattice structure, fabricated by any of the methods referred to above, is amenable to high porosities; over 60%, and preferably over 85%. High porosity is advantageous for use in wet conditions. This compares favourably to the 40% to 45% porosity achieved using granular matrix materials, or porous blocks sintered from granular matrix material. Advantageously, the higher porosity provided by the substantially open-cell micro-lattice structure of the porous material more closely matches the porosity of soil-free substrates, and has similar hydraulic properties.

The skilled person would understand that if granular or sintered porous portions (with porosities below 50%) are used in soil-free media, the hydraulic properties of these portions are compromised. For example, these granular or sintered porous portions may show wetting/drying hysteresis and slower response times. By contrast, the high porosity open-cell structure of the porous portion of the embodiments being described allows appropriate hydraulic conductivity to be maintained and allows for rapid response times.

Control of pore size is also possible by the above methods, with pores generally ranging from 1 μm to 500 μm across; pore sizes toward the larger end of the range are preferable for hydrophilic materials operating at water potentials close to 0 kPa, i.e. operating in very wet conditions. Preferably, the porous material contains pores with a range of diameters. The range of diameters may be from 1 μm to 500 μm. Advantageously, having a range of pore diameters increases the range of water potentials over which the sensor can operate, as pore size is a factor in determining the water potential at which a pore will drain.

Advantageously, the combination of high porosity and an open cell structure facilitates the escape of air within the porous material portions on the ingress of water.

In some embodiments there may be provided a single porous material portion. However, in other embodiments there may be provided more than one porous material portion.

In embodiments where there is more than one porous material portion, one or more dimensions of the porous material may be varied between portions. For example, the pore size may be varied between portions. Such embodiments are believed to be advantageous in that the operating range of the sensor is increased.

Additionally, the portions of porous material may be separated, optionally by a circuit board.

Additionally or alternatively, the portions of porous material may be made of different materials. This may facilitate the creation of elements with differing ranges of pore sizes.

In some embodiments an adhesive may be used to secure the or each portion of porous material in place. In particular, the adhesive may be arranged to secure the or each portion of porous material to one another.

Typically, should an adhesive be used, it will be used in a region of the porous material removed (i.e. distant) from the electrodes.

Preferably, the porous material portion in any embodiment is arranged to allow simple attachment to the sensor body, removal and replacement. In some embodiments, a fastening hole is provided on the sensor, to which the porous material can be attached using a clip or screw or other fastening means. In other embodiments, other attachment means known in the art may be provided, for example an elongate fastener, elasticated band or net, or an adhesive.

In various embodiments, the porous material portion is sufficiently compliant to fill air gaps between the porous material and the electrodes, and additionally to give good contact with a material into which, in use, it is inserted.

In some embodiments, the porous material may contain recesses in which the electrodes are provided and the porous material is conveniently sufficiently compliant to ensure contact with the electrodes.

In various embodiments, a cage is provided to enclose the porous material. The cage may improve attachment of the portion of porous material to the body of the sensor. A fixing hole can be used to facilitate attachment of the cage to the body of the sensor using a clip or screw, or any other attachment means known in the art may be used as previously discussed for the porous material.

The cage advantageously provides physical support and protection for the portion of porous material. The cage is designed to not significantly impede water flow between the portion of porous material and the material into which, in use, the sensor is inserted.

The skilled person will understand that the cage could be made of a plastics material, amongst other materials, but is preferably made of metal or another electrically conductive material. Stainless steel may be favoured due to its conductivity and corrosion resistance.

In embodiments with electrically conductive cages, the cage additionally acts as a Faraday cage for the portion of porous material. This advantageously reduces stray-field related errors from leakage of the electric field into the surrounding area and/or from external fields.

In some embodiments, the cage is in the form of a thin-walled cup. The thin-walled cup may comprise circular holes in at least one of the wall and base to facilitate water movement. In alternative embodiments, the cage may comprise crossing struts, a rigid net or a mesh, amongst other possibilities apparent to the skilled person.

In some embodiments, the electrodes are provided as a circuit board comprising one or more tracks. In circuit boards according to known fabrication techniques, variations in the thickness of tracks of up to 10 μm have been recorded. Also, in such circuit boards, the weave of fibres within the fabric of the board can also lead to variations in the surface of the board. As such, an advantage of embodiments that use a compliant material for the porous material portions is that such variations can be absorbed thus helping to remove pockets that might otherwise hold water.

Typically, the or each track of the circuit board is arranged to pass an electric current.

At least one track, and typically two tracks, provided on the sensor may be arranged to form a plate of a capacitor. As such, sensing electronics may be arranged to measure a variation in capacitance of the sensor. The capacitance of the sensor may be arranged to be caused to vary by the moisture content of the surrounding test medium, such as soil.

In alternative, or additional, embodiments the electrodes may be provided as one or more pins. Typically, a plurality of pins is provided.

In some embodiments the matric potential sensor may be a dielectric tensiometer.

According to a second aspect of the invention, there is provided an irrigation system comprising a sensor according to the first embodiment of the invention, which is preferably automated. A level of watering can be triggered, increased, decreased or stopped in response to measurements taken by the sensor. Irrigation control employing feedback from such a sensor may help to avoid plant water stress resulting from low hydraulic conductivity conditions, for example as found in gap-graded media such as coir once the larger pores have been drained.

According to a third aspect of the invention, there is provided a measurement kit comprising one or more sensors and multiple porous material portions according to the first aspect of the invention, preferably with the porous material portions having a range of pore sizes to facilitate water potential measurement over a wide variety of water content conditions.

According to a fourth aspect of the invention, there is provided a moisture content meter comprising at least one sensor according to the first aspect of the invention and at least one of the following:
(i) a human-readable output and means for displaying the output; or
(ii) a machine-readable output and means for communicating the output to a machine capable of reading the output.

In some embodiments, the output is communicated to a computer or other device electronically using a wireless signal. In other embodiments, a wired connection is used. In alternative or additional embodiments, the output may be displayed on a screen which forms part of the sensor.

According to a fifth aspect of the invention, a method of fabricating a matric potential sensor is provided. The method comprises inserting one or more portions of an open-cell, hydrophilic micro-lattice matrix around the electrodes of the sensor.

The skilled person will appreciate that features described in relation to any one of the above aspects of the invention may be applied, mutatis mutandis, to any other of the aspects of the invention.

There now follows by way of example only a detailed description of embodiments of the present invention with reference to the accompanying drawings in which:

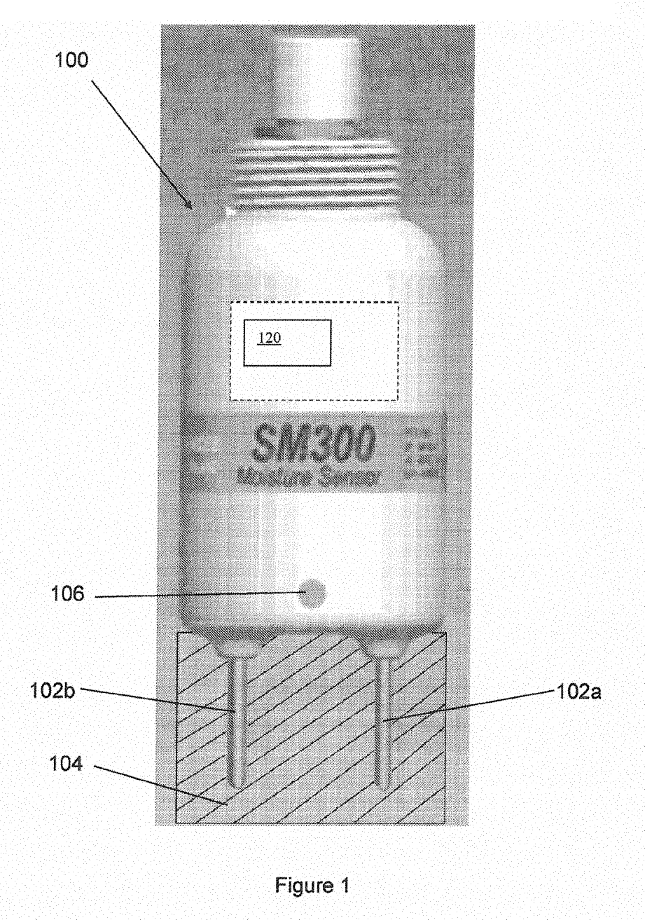

FIGS. 1 shows a sensor of an embodiment;

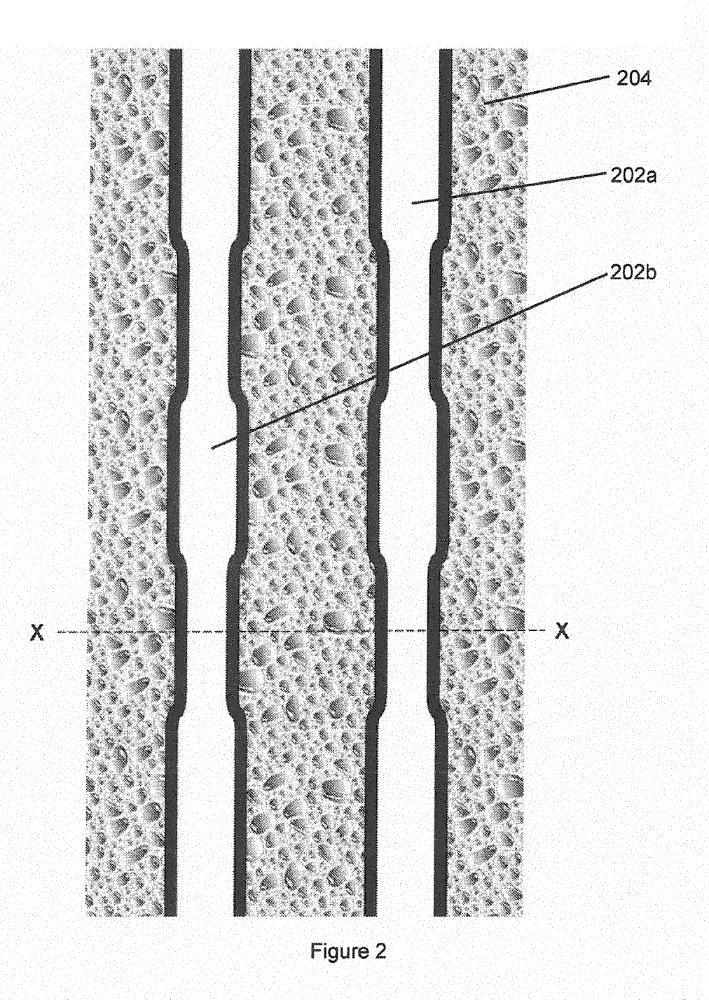

FIG. 2 shows a section through electrodes and a matrix of an embodiment similar to that shown in FIG. 1;

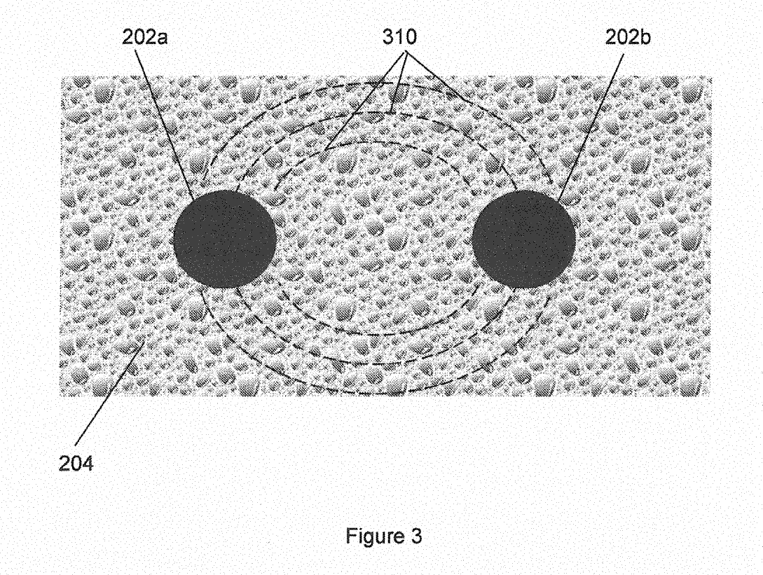

FIG. 3 shows a section along line X-X of FIG. 2;

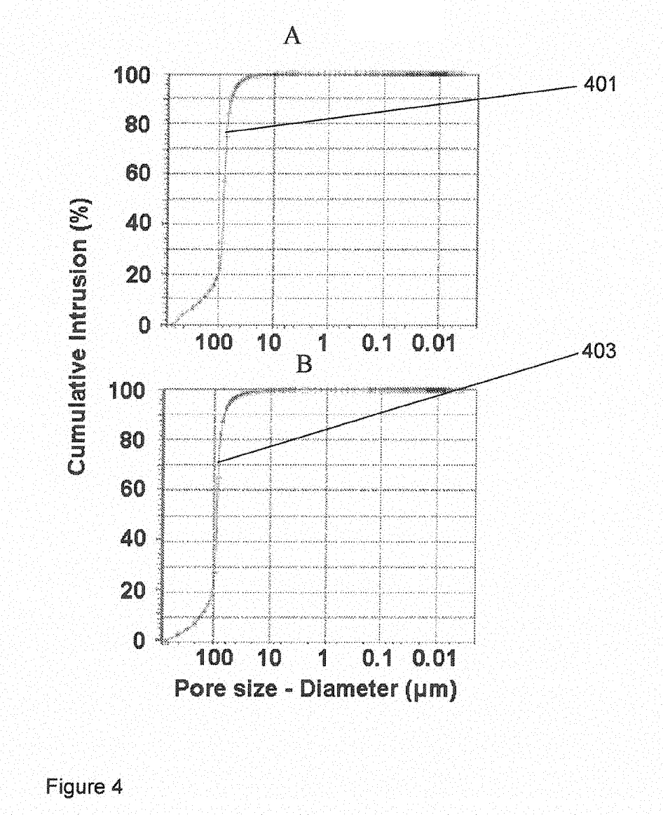

FIG. 4 shows experimental results showing pore-size distribution characteristics of a porous matrix of an embodiment;

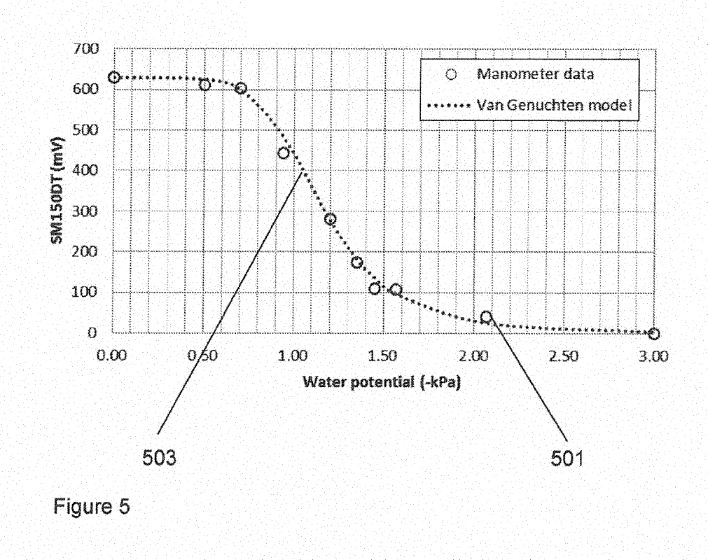

FIG. 5 shows experimental results showing the response performance of a sensor of an embodiment;

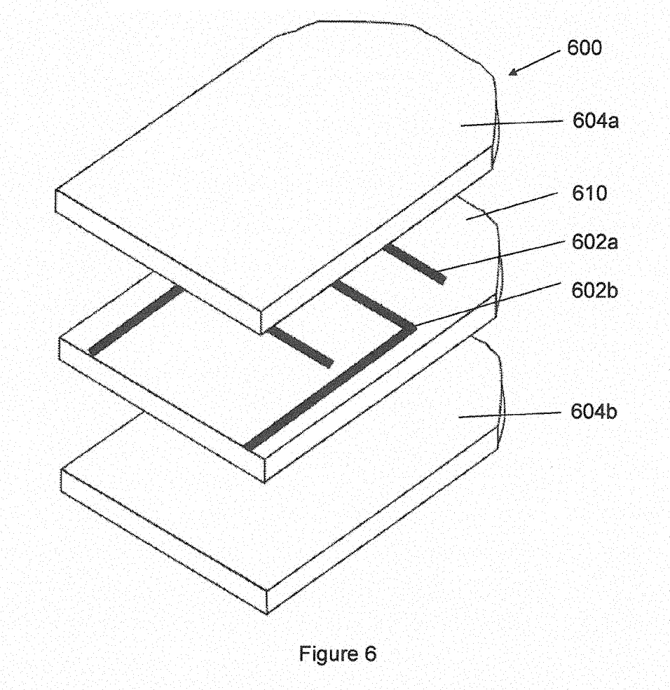

FIG. 6 shows an exploded perspective view of a sensor of an alternative embodiment;

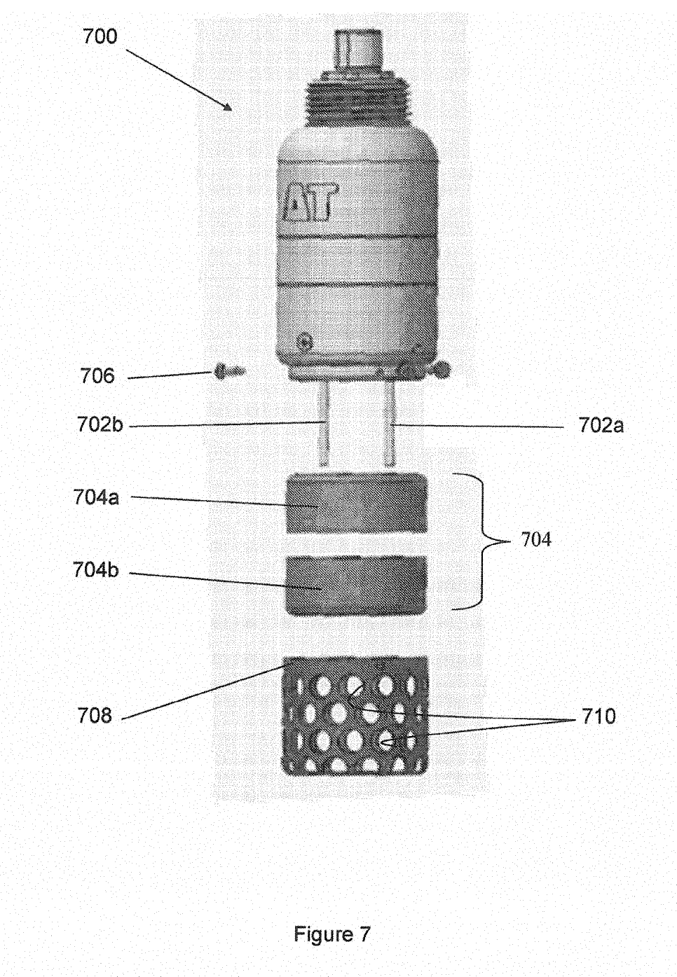

FIG. 7 shows a sensor of an embodiment wherein optional cage is provided;

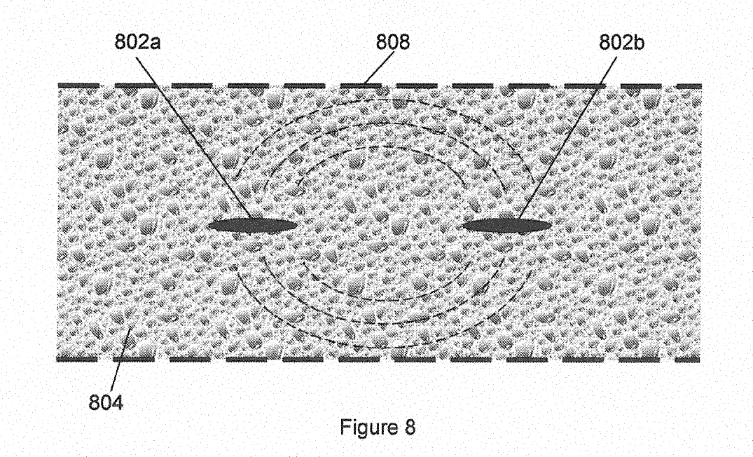

FIG. 8 shows a section through the electrodes and matrix for an embodiment in which the electrodes are over-printed;

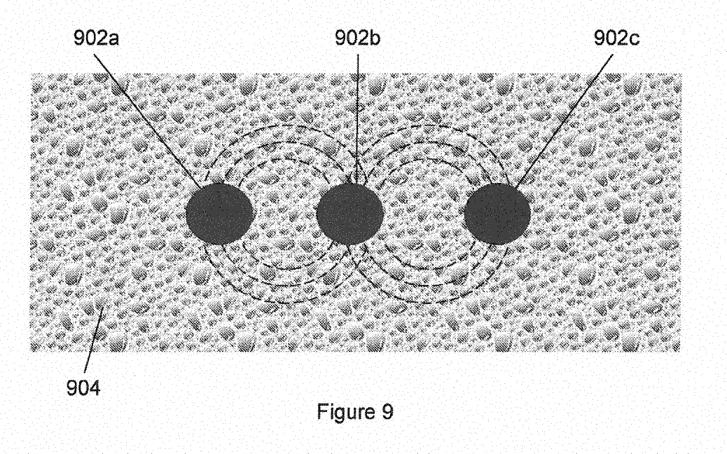

FIG. 9 shows a section for an embodiment in which a three-electrode arrangement is used; and

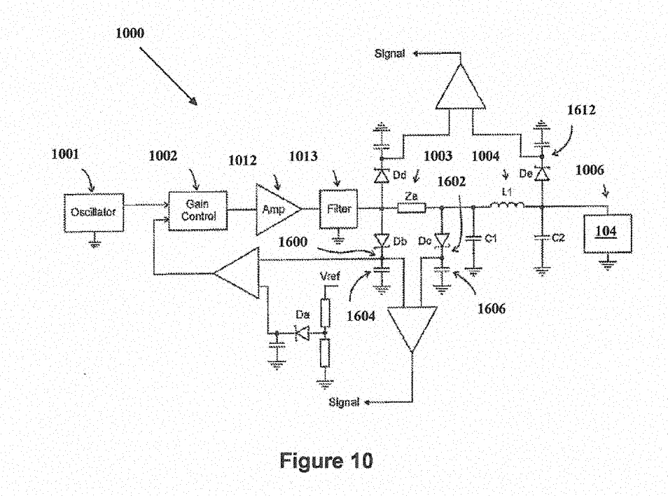

FIG. 10 (Prior Art) shows example sensing electronics for use in the embodiments described with reference to FIGS. 1 to 9.

Looking at FIG. 1, the sensor 100 has two electrodes 102a, 102b, which are in the form of pins, which pins may be thought of as forming sensing elements. A matrix is provided by a portion of porous material 104 is provided with two holes (not shown on FIG. 1), which in the embodiment being described are blind, of complementary shape and size to the two electrodes 102a, 102b. In the assembled sensor 100 the two electrodes 102a, 102b are inserted into the two holes such that the porous material surrounds the electrodes 102a, 102b and the electrodes 102a, 102b extend down into the porous material 104.

Each of the electrodes 102a, 102b provides the plate of a capacitor, the capacitance of which varies according to the dielectric constant of the portion of porous material 104 adjacent the electrodes. The dielectric constant is affected by the amount of water held within the porous material 104, which in turn is affected by the water potential of water surrounding the porous material.

In other embodiments, the two electrodes 102a, 102b can be forced into the porous material 104 such the electrodes create complementary holes therein as the electrodes are inserted.

Fixing hole 106 is provided in the body of the sensor 100 to facilitate attachment of the portion of porous material 104.

The portion of porous material 104 in the present embodiment is a substantially open-cell hydrophilic micro-lattice matrix. For the avoidance of doubt, hydrophilic materials are wettable and have a water contact angle of smaller than 90°. The micro-lattice of this embodiment is a three-dimensional (3D) network structure consisting of webs of material, wherein the pore diameters are on the order of 1 μm to 500 μm. A micro-lattice matrix made in accordance with this embodiment of the invention was found to give contact angles in the range of 10° to 85° , and preferably in the range of 60° to 75°.

The porous material 104 has a porosity of greater than 60%, and preferably greater than 85%.

In the embodiment being described, the porous material 104 is polymeric and in particular is polyurethane. Thus, the porous material 104 is also compliant.

In other embodiments, other plastics materials, ceramics and composite materials could be used. Indeed, in some embodiments, more than one of these materials may be used.

In the embodiment being described, the micro-lattice matrix of the porous material 104 is provided by a reticulated and compressed foam. An advantage of reticulation and compression is that the porosity and open-cell nature of the initially-formed foam are increased and the pore size maybe controlled.

FIG. 2 shows a cross-section through the porous material 204 in an embodiment similar to that shown in FIG. 1 but wherein the electrode pins 202a, 202b are of varying cross-section rather than constant cross-section. In the embodiment shown, the shape of the portion of porous material 204 demonstrates the compliance of the matrix, conforming to the shape of the pins 202a, 202b.

FIG. 3 shows a cross-section through the porous material 204 taken along line X-X in FIG. 2. Electric field lines 310 are shown around the electrodes 202a, 202b, within the porous material 104. It should also be noted that the porous material 104 is in close proximity to the pins 202a, 202b such the pins 202a, 202b are in substantial contact with the porous material 204.

The sensor 100 comprises sensing electronics 120, which may be as described in EP 1 836 483, which is hereby incorporated by reference, and arranged to inject a signal into the matrix provided by the portions of porous material 104. The sensing electronics 120 may be provided anywhere within the body of the sensor 100. The skilled person will understand that other locations of the sensing electronics 120 are possible. For example, the sensing electronics 120 could be located on the pins 102a, 102b, which may be thought of as sensing elements.

FIG. 10 shows an example of sensing electronics 1000 that may be used, and as is described in more detail in EP 1 836 483. It will be seen that an oscillator 1001, a gain control 1002, sensing impedance Za 1003 and a complex impedance 1004 (C1, L1, C2) are provided. The circuit shown may also measure the differential signal between the point between the sensing impedance and the oscillator 1001 and the output side of the complex impedance 1004.

Thus, the sensing electronics 1000 measures a differential signal measured across sensing impedance Za 1003. Peak detector circuits 1600, 1602, comprising Schottky diodes Db and Dc and charging capacitors 1604, 1606, as will be readily appreciated by the skilled person.

Using the sensing electronics 1000 shown, it is possible to detect changes in moisture content of the porous material 104 from changes in the amplitude of the signal (for example using the peak detector circuits 1600, 1602).

Thus, it will be seen that the sensing impedance Za 1003, is provided as a potential divider with the impedance formed by the complex impedance 1004 (C1, L1, C2) and the porous material 104. Here, it will be appreciated that because the sensor is a matric potential sensor, it is the moisture content of the porous material 104 that is being directly measured by the sensing electronics 1000 rather than that of the surrounding medium. However, the moisture content of the porous material 104 is related to that of the surrounding medium. Looking at the embodiment of FIG. 7, a signal will typically be injected via one of the pins 702a, 702b whilst the other of the pins 702a, 702b will be grounded.

The impedance of at least some of the matrix, the sensing electronics and the sensing elements form a potential divider and the sensing electronics are arranged to measure a change in a parameter of that potential divider. The skilled person will appreciate that the impedance of the matrix varies as the moisture content of the matrix varies.

FIG. 4 shows experimental mercury intrusion porosimetry results concerning pore-size distribution characteristics of a sample of porous matrix material suitable for use in the embodiments described herein. Cumulative intrusion is plotted as a percentage against pore size (diameter) in micrometres, which is shown on a logarithmic scale. Larger pores are filled more easily; the pressure must be increased to force the mercury into smaller pores. Pore size is therefore calculated from the pressure required to force the mercury into the pores.

Two samples were tested data for samples A and B are shown by lines 401, and 403 respectively.

Porosity data extracted from the mercury intrusion porosimetry data plotted in FIG. 4 is tabulated in Table 1, below. The foam, B, used for sensor trails for an embodiment had a porosity of 85.1% and a mean pore diameter of 47.4 μm.

Sample A is a polyurethane foam based on a hydrophobic polyol and sample B is a polyurethane foam based on a hydrophilic polyol the foams have undergone a substantially identical reticulation and compression processes. These examples are provided for illustrative purposes only; the invention is not to be limited by the type of polyol employed, nor by the type of reticulation or compression process

TABLE 1

| | Sample | |
|---|---|---|
| Property | A | B |
| Total Intrusion Volume (mL/g) | 4.1916 | 4.365 |
| Total Pore Area (m²/g) | 0.216 | 0.368 |
| Median Pore Diameter (Volume, μm) | 79.2793 | 87.7676 |
| Average Pore Diameter (4 V/A) μm | 77.4656 | 47.4098 |
| Bulk Density at 0.31 psia (g/mL) | 0.2 | 0.195 |
| Apparent (skeletal) Density at 5.9953.48 psia (g/mL) | 1.2363 | 1.309 |
| Porosity (%) | 83.8246 | 85.1056 |

The manufacturing process of the polyurethane foam, including its raw material constituents, allows for adjustment of the pore-size distribution, whilst allowing the porosity to remain largely unchanged. It can be seen from Table 1 that the average (mean) pore diameters of A and B differ significantly, whereas the total porosities are very similar.

In use, the sensor 100 is inserted into a medium to be tested, such as a soil, such that the portion of porous material 104 is completely surrounded by the test medium.

The sensor is used to measure water potential in the medium by measuring the potential energy of water which is in equilibrium with water in the medium. In accordance with the Second Law of Thermodynamics, connected systems with different energy levels move toward equilibrium; therefore once the porous material 104 comes into hydraulic contact with the medium under test, the water potential within the porous material 104 equilibrates with that in the test medium. Equilibration times vary depending on the applied pressure gradient and the hydraulic conductivity of the porous material, being shorter in wetter media. A measurement of the water potential within the porous material 104 should therefore be a measurement of the water potential in the medium once the potentials have reached equilibrium. Use of a hydrophilic, high porosity open-cell matrix, as the porous material, reduces this equilibration time by increasing the ease with which water can move into and out of the porous material 104 so facilitating rapid measurements.

Medium water content and water potential are related by a moisture characteristic curve, which is unique to the material. Prior to use, the water content and corresponding potential of the porous material 104 is measured accurately to generate a calibration curve. This calibration is independent of medium type so can be used to infer the water potential from readings taken using the tested porous material portions in any medium.

FIG. 5 shows experimental results 501 showing the response performance of a sensor of this embodiment and provides a moisture characteristic curve. Response of the sensor (for sensor SM150DT) in mV is plotted against water potential in kPa. The data presented in FIG. 5 demonstrate a marked change in sensor response with water potential over the range of −0.6 to −3 kPa the sensor response 501 changes from over 600 mV to 0 mV over this range of water potentials. A voltage change of this size is easily measureable and the smooth curve between extreme values facilitates interpolation between the measured points 501.

The Van Genuchten Model for water retention in soil was used to confirm that the sensor response followed expected trends. This model is defined by the following equation (from van Genuchten M. Th., 1980, A closed-form equation for predicting the hydraulic conductivity of unsaturated soils, Soil Sci. Soc. Am. J., Vol. 44, pp 892-898), wherein θ is water content at water potential h, θₛ and θᵣ are saturated and residual water contents respectively and α, n and m are empirical coefficients.

θ=(θₛ−θᵣ)[1+(αh)ⁿ]^{-m}+θᵣ

where:

m=1−1/n

The sensor response 501 is represented by θ and the values for θ, and θᵣ and for the empirical coefficients a, n and m are provided in Table 2, below.

TABLE 2
Van Genuchten parameters

| | |
|---|---|
| θₛ (mV) | 629.000 |
| θᵣ (mV) | 0.000 |
| α | 0.905 |
| n | 6.081 |
| m | 0.836 |

As shown in FIG. 5, the measurement data 501 are in good agreement with the theoretical model 503.

The large pore sizes and the highly porous, open-cell structure of the porous material allow embodiments having this structure to operate over a water potential range of substantially 0 to −10 kPa. This is advantageous in that sharp decreases in hydraulic conductivity have been observed over this range (supra).

The high porosity and large pore sizes of the porous portion 104 of the embodiment being described enable water potential to be measured accurately even under wet conditions. Compliance of the porous material 104 can enhance reliability by filling gaps adjacent the electrodes 102a, 102b in which water might otherwise gather and distort measurements.

The following embodiments demonstrate variations on the above embodiment. Like parts have been reference with similar reference numbers with the two rightmost digits remaining constant but with the left most digit amended.

FIG. 6 shows a second embodiment in which the porous material portion 604a, 604b is split into two such that a portion is provided on each side of a sensor element which in this case is a circuit board 610. As with the pins 102a, 102b the circuit board may be thought of as being sensing elements. The circuit board 610 is provided with printed tracks thereon which provide two electrodes 602a, 602b. Like the pins 102a, 102b, the tracks of the circuit board 606 can be used to measure the moisture content within the porous portions 604a, 604b.

The sensing electronics may be mounted off the circuit board 610 as is the case in the embodiment being described in relation to FIG. 6, but in other embodiments the sensing electronics may be mounted on the circuit board 610. Indeed, it would be possible for some of the sensing electronics to be mounted upon the circuit board 610 and some of the sensing electronics to be mounted off the circuit board 610.

In the embodiment being described, the porous material 604a, 604b is a 3D printed structure. The structure consists of slender webs or linked struts, and may be described as trabecular in nature.

FIG. 7 shows a sensor 700 of a further embodiment, in an exploded form, in which the matrix is provided by two portions 704a, 704b of porous material surrounding the electrodes 702a, 702b. In addition, the sensor 700 comprises a cage 708 which surrounds the first 704a and second 704b portion of porous material. The portions of porous material 704a, 704b are arranged, in use, to be adjacent to each other. The skilled person will understand that the portions of porous material 704a, 704b are preferably in direct contact with each other, but that there may be a gap between them. Thus, the matrix which is comprised, in this embodiment, by the two portions of porous material 704a, 704b may also include a gap therebetween.

Portions of porous material 704a and 704b are structured to have differing pore size ranges so that the overall pore size range is larger than that in either portion of porous material alone. This has the advantage of increasing the overall operating range of the sensor 700.

The cage 708 improves attachment of the portion of porous material 704a, 704b to the body of the sensor 700. Fixing hole 706 can be used to facilitate attachment of the cage 708 to the body of the sensor 700.

The cage 708 provides physical support and protection for the portion of porous material 704a, 704b. The cage 708 is designed to not significantly impede water flow between the portion of porous material 704a, 704b and the material into which, in use, the sensor 700 is inserted having a plurality of holes 710 through the surface thereof. In the embodiment shown, it can be seen that the holes 710 occupy roughly 60% of the surface of the cage 708. In other embodiments, the holes in the surface of the cage 708 may occupy roughly in the range of 40% to 80% of surface. In further embodiments, the holes in the surface of the cage 708 may occupy roughly in the range of 50% to 70% of the surface. The skilled person will appreciate that numbers in between the listed percentages may also be possible.

In the embodiment being described, the cage 708 is made of stainless steel, and thereby the cage 708 also acts as a Faraday cage 708 for the sensor 700 retaining electric field from the electrodes 702a, 702b within the cage 708 and preventing leakage of the field into the medium in which, in use, the sensor 700 is being used.

The skilled person will appreciate that such a cage 708 could equally be used, mutatis mutandis, in the embodiments of the invention shown in FIG. 1 and FIG. 6.

In the embodiment shown in FIG. 7, the cage 708 is in the form of a thin-walled cup with circular holes in the walls and base to facilitate water movement. In some embodiments, there is a larger hole in the cup in the region where the hydraulic connectivity to the medium is highest on insertion into the medium. The region with the highest hydraulic connectivity to the medium is the bottom of the cup in some of these embodiments.

FIGS. 8 and 9 show equivalent cross-sections to that shown in FIG. 3 for two further embodiments of the invention wherein the arrangements of electrodes 802a, 802b, 902a, 902b, 902c differ.

The electrodes 802a, 802b in FIG. 8 are flexible printed circuit boards (PCBs). They are arranged to be over-moulded; i.e. a 3D structure can be printed on to one or both sides of the PCB. This process is also referred to as over-printing and can improve surface contact with the electrodes. In alternative embodiments, a thin film or foil is used in place of the PCBs.

In this embodiment, a conductive cage 808 is also present, surrounding the portion of porous material 804.

The embodiment of FIG. 9 uses three electrodes 902a, 902b, 902c within the portion of porous material 904. Although a cage could be used in other embodiment using this electrode configuration, the three electrode arrangement advantageously provides additional screening which helps to minimise stray-field related errors.

1. A matric potential sensor comprising sensing electronics arranged to measure a change in a signal injected into a matrix via one or more sensing elements, wherein the matrix comprises at least one portion of a substantially open-cell hydrophilic micro-lattice matrix arranged to absorb moisture from a medium into which, in use, it is inserted.

2. The sensor of claim 1 which substantially open-cell hydrophilic micro-lattice matrix has a porosity of over 60%.

3. The sensor of claim 1 wherein the sensor is arranged to absorb moisture from a soil-free medium.

4. The sensor of claim 1 wherein the substantially open-cell hydrophilic micro-lattice matrix is a reticulated foam.

5. The sensor of in which the impedance of the matrix together with an impedance within the sensing electronics form a potential divider and the sensing electronics is arranged to measure a parameter of that potential divider.

6. The sensor of claim 1 in which the sensing elements comprise one of:
i) a set of two or more pins; and
ii) a circuit board on which one or more tracks are provided.

7. The sensor of claim 1 wherein the matrix is present in the form of one or more portions of porous material.

8. The sensor of claim 1 wherein the matrix is substantially enclosed in a cage.

9. The sensor of claim 8 wherein the cage is arranged to support the matrix.

10. The sensor of claim 8 wherein the cage is electrically conductive.

11. (canceled)

12. The sensor of claim 1 wherein the matrix comprises pores substantially in the range of between 1 μm and 500 μm in diameter.

13. The sensor of claim 1 wherein the matrix is made from polyurethane.

14. The sensor of claim 1 wherein the matrix is trabecular in form.

15. The sensor of claim 1 wherein the matrix is a foam.

16. (canceled)

17. The sensor of claim 1 wherein the matrix is fabricated from a compliant material.

18. The sensor of claim 1 wherein matrix is composed of at least two portions of porous material and the pore size distributions differ between the portions of porous material.

19. (canceled)

20. The sensor of claim 1 wherein the matrix is formed by a solid freeform fabrication and, rapid prototyping technique.

21. A kit comprising at least one sensor of claim 1 and porous material portions suitable for use in the sensor.

22. A moisture content meter comprising at least one sensor of any claim 1 and at least one of the following:
(i) a human-readable output and means for displaying the output; or
(ii) a machine-readable output and mean for communicating the output to a machine capable of reading the output.

23. A method of fabricating a mage potential sensor of claim 1 wherein the method comprises inserting one or more portions of the open-cell, hydrophilic micro-lattice matrix adjacent at least one sensing element.
